(19) United States
(12) Patent Application Publication
(10) Pub. No.: US 2025/0144170 A1
(43) Pub. Date: May 8, 2025

(54) METHOD FOR SUSTAINED DRUG DELIVERY FOR WOUND HEALING USING SILK HYDROGEL AND PRODUCT THEREOF

(71) Applicant: AASYA HEALTH CARE PRIVATE LIMITED, Hyderabad (IN)

(72) Inventors: Vikas Sahu, Prayagraj (IN); E Kranti Kiran Reddy, Hyderabad (IN)

(73) Assignee: AASYA HEALTH CARE PRIVATE LIMITED, Hyderabad (IN)

(21) Appl. No.: 18/836,489

(22) PCT Filed: Feb. 7, 2023
(86) PCT No.: PCT/IB2023/051062

§ 371 Date: Aug. 7, 2024

(30) Foreign Application Priority Data
Feb. 7, 2022 (IN) 202241006371

(51) Int. Cl. A61K 38/17; A61K 9/00; A61K 9/06; A61K 9/19; A61K 31/65

(52) U.S. Cl. CPC A61K 38/1767; A61K 9/006; A61K 9/06; A61K 9/19; A61K 31/65

(57) ABSTRACT

The present invention provides a silk fibroin-based hydrogel and antibiotic composition consists of 5% silk fibroin embedded with 0.4-2% antibiotic, more specifically, tetracycline or doxycycline, and the antibiotic used to prepare the silk fibroin-based hydrogel is super saturated solution, i.e., 100% concentration of antibiotic in de-ionized water. The invention also provides a method to prepare silk fibroin-based hydrogel and antibiotic composition comprising steps of: preparing 5% silk fibroin solution and maintaining at 16-21° C.; adding saturated antibiotic solution in the silk fibroin solution to form silk fibroin-based hydrogel; stabilizing the hydrogel at 16-21° C.; and lyophilising the stabilized hydrogel at 4-6° C. to form a dry hydrogel silk fibroin matrix with drug embedded in it. Further, the invention provides composition of the drug-silk fibroin-based hydrogel formulation and method for using it for wound healing of tooth extraction socket.

### PRIORITY PARAGRAPH

The Application claims priority to PCT application PCT/IB2023/051062 filed on 7 Feb. 2023 titled “Method for sustained drug delivery for wound healing using silk hydrogel and product thereof” that claims priority to the Indian provisional application 202241006371 Titled “Method for sustained drug delivery for wound healing using silk hydrogel and product thereof” filed on 7 Feb. 2022. Both priority applications are fully incorporated herein by reference.

### FIELD OF THE INVENTION

The present invention relates to a method for sustained drug delivery for wound healing using silk hydrogel and product thereof. More specifically, a method to manufacture silk hydrogel formulation capable of releasing antibacterial and osteoinductive agent at a sustained and controllable rate to facilitate wound healing in tooth extraction socket.

### BACKGROUND OF THE INVENTION

Dental treatment leading to extraction includes acute/chronic pulpitis, periapical inflammation/abscess, severe periodontitis and alveolar bone loss leading to mobility of teeth. The associated bacterial infection is specifically localized in nature and confined to dental pulp tissue to periapical region within alveolar bone. The source of infection, which includes decayed tooth or root once removed, will heal the wound without secondary complication in a healthy individual. Prescription of systemic antibiotics after tooth extraction is the most common practice by dentists around the globe. The most common reason for this practice is to prevent secondary infection in the wound that has been created after extraction.

Generally, there is no need of antibiotics if the infection is localised and there is no abscess or pus formation, which is usual in conservative tooth extraction cases. But considering the general clinical scenario, where strict sterilisation protocol in the clinic is limited and poor oral hygiene practice of population (patients), dentist prefer to give systemic antibiotics orally which include Amoxicillin, Amoxicillin+Clavulanic acid, Fluroquinolones, Tetracycline/Doxycycline etc. It is also administered that use of such systemic antibiotics cause side effects like gastric irritation, nausea, vomiting, and diarrhoea, which are uncomfortable to the patients. Further, excessive use of antibiotics leads to antibiotic resistance.

Very often, a dentist uses any surgical dressing in such cases as surgical dressing materials require surgical retrieval and have no inherent antibiotic activity. Further, It is well noted that there are no specific surgical dressing or gauges available for tooth extraction or wound management cases. Hence, there remains a need for compositions comprising a natural polymeric medium that offers a medically-relevant, biocompatible, and mechanically viable structure for drug delivery to the target delivery area to effectively prevent or treat an infection.

### PRIOR ART

CN110090320A provides a conical structured teeth socked recovery support with antibacterial activity. The conical structure is solidified by repair materials to be formed, and one layer of collagen dense film is adhered at the cone bottom of the conical structure, described Repair materials include collagen-hydroxyapatite composite material, collagen, minocycline hydrochloride, wherein collagen-hydroxyapatite is multiple Condensation material, collagen, broad spectrum antibiotic mass ratio be 1˜6:0˜6:0.1˜1.5, and collagen is not 0. The collagen dense film to carry out extruding acquisition after collagen solution freeze-drying. The method involves: vacuum freezing collagen solution to obtain collagen sponge, densification of collagen sponge to obtain Collagen membrane; preparing Collagen-hydroxyapatite suspension using collagen, calcium hydroxide and phosphoric acid, adding collagen solution and the material containing broad spectrum antibiotic to obtains mixed liquor; injecting Mixed liquor in mold, so that mold is filled full mixed liquor, is covered mold mouth using dense collagenous film, carry out Vacuum freeze drying. The mold is to contain the tool with the consistent pit of teeth socked shape.

CN111849013A provides a preparation method of a nano hydroxyapatite-silk fibroin mineralized scaffold. The steps involve: immersing the silk fibroin bracket in mineralized liquid for reaction to obtain the silk fibroin bracket; the mineralized liquid is a mixed aqueous solution of ethylenediaminetetraacetic acid (conc. 0.2-0.3 mol/L), and calcium sodium and sodium dihydrogen phosphate (conc. 0.1-0.2 mol/L). The silk fibroin bracket is immersed in the mineralized liquid for reaction under the reaction conditions of 120-130° C. and 1-3 atm for 12-24 hours, preferably, the reaction condition is 121° C. and 2 atm for 24 h. Further, it involves freeze drying at −80° C. for 12 h and then freeze drying at −50° C. This requires expensive chemicals and maintenance of very high temperatures and freezing temperatures for at least 24 hrs. This significantly increases the cost of production.

The use of silk fibroin and its use in wound healing have been well documented. However, still the existing technology has a lot of scope for optimisation of conditions to provide better retention and sustained delivery of the drug for facilitated wound healing in tooth extraction socket. Moreover, mouth has a very dynamic environment therefore application of any formulation which is sustained and uninterrupted is very difficult.

### OBJECTS OF THE INVENTION

Accordingly, to overcome the drawbacks of the prior art the main object of the present invention is to provide silk fibroin-based hydrogel formulation capable of sustained and controlled release of drug more particularly antibacterial and osteo-inductive agent at a sustained and controllable rate, more particularly formulated for wound healing of tooth extraction sockets.

Another object of the present invention is to provide an affordable solution for the localized delivery of antibiotic drug at the site of tooth extraction which can be easily placed by a dentist/medical practitioner.

Yet another object of the present invention is to provide a silk fibroin-based hydrogel formulation capable of sustained release of antibiotic drug at effective for 3-5 days to prevent secondary infection at the site of tooth extraction wound, leading to decreased used of systemic antibiotics.

Yet another object of the present invention is to provide a silk-based hydrogel formulation capable to form a suitable microenvironment to control bacterial secondary infection and augment the alveolar bone regeneration.

### SUMMARY OF THE INVENTION

In carrying out the above objects of the present invention, the present invention provides a method for sustained drug delivery for wound healing using silk hydrogel and product thereof for wound healing of tooth extraction socket. More specifically, the invention provides a silk fibroin-based hydrogel formulation capable of releasing antibacterial and osteoinductive agent at a sustained and controllable rate.

In the main embodiment of the present invention, the invention provides a silk fibroin-based hydrogel and antibiotic composition for wound healing of tooth extraction sockets, wherein, the silk fibroin-based hydrogel and antibiotic composition consists of 5% silk fibroin embedded with 0.4-20% antibiotic, more specifically, tetracycline or doxycycline, and the antibiotic used to prepare the silk fibroin-based hydrogel is super saturated solution of antibiotic, more specifically, 100% concentration (100 mg/ml) of antibiotic. The hydrogelation process is enabled by addition of 100% concentration (100 mg/ml) of antibiotic to the 5% silk fibroin.

The silk fibroin-based hydrogel enables drug delivery tailored for controlled release of antimicrobials, which also propagates the regeneration of injured cells. The hydrogel comprises of lyophilised Doxycycline, or Tetracycline on a silk fibroin support structure. The lyophilised gel removes the water molecules and increases the shelf life.

In another embodiment the present invention, the invention provides a method for formulation of silk-based hydrogel formulation for wound healing of tooth extraction sockets, the method of formulation comprising steps of:
a) preparing 5% silk fibroin solution;
b) taking 500 μl of prepared 5% silk fibroin solution and maintaining at 16-21° C.;
c) preparing a super saturated solution of tetracycline/doxycycline drug by adding 100 mg of drug in 1 ml of de-ionized water and vortexing it for 30-60 sec to prepare a 100% concentration drug solution;
d) adding saturated tetracycline/doxycycline solution in the 5% silk fibroin solution to form a silk fibroin-based hydrogel;
e) stabilizing prepared hydrogel at a temperature of 16-21° C. for first 8 hours;
f) subsequently by refrigerating at 4-6° C. for next 3 days (72 hours); and
f) lyophilising the stabilized prepared hydrogel to form a dry hydrogel silk fibroin matrix with drug embedded in it.

The prepared silk-based drug hydrogel shows high amount of drug release for first 3-5 days, and subsequently shows a low drug release for next 15 days.

In another embodiment of the present invention, a method for using a drug-silk fibroin-based hydrogel formulation for wound healing of tooth extraction socket is provided, the method comprising:
cleaning the extraction socket post tooth extraction;
normalizing the lyophilised silk drug hydrogel using de-ionized water to introduce flexibility;
reshaping the normalized lyophilised silk drug hydrogel similar to extracted tooth root profile;
placing the normalized lyophilised silk drug hydrogel in the extraction socket post tooth extraction; and
retaining the normalized lyophilised silk drug hydrogel in the socket embedded in the blood clot to enhance wound healing and osteoinduction;
wherein,
the silk-based drug hydrogel locally releases the drug (tetracycline/doxycycline) in controlled manner in microbicidal concentration (localised concentration has to optimised: MIC is 1.2 ul/ml of body fluid) for first 4-5 days; and
the silk-based drug hydrogel locally releases the drug at sub-microbicidal concentration for next 15 days.

In another embodiment of the present invention, there is provided a novel silk-based hydrogel system wherein the biological degradation of same was enhanced by using proteinase-K.

### BRIEF DESCRIPTION OF THE DRAWINGS

The object of the invention may be understood in more details and more particularly description of the invention briefly summarized above by reference to certain embodiments thereof which are illustrated in the appended drawings, which drawings form a part of this specification. It is to be noted, however, that the appended drawings illustrate preferred embodiments of the invention and are therefore not to be considered limiting of its scope, for the invention may admit to other equally effective equivalent embodiments.

FIG. 1 is a schematic view of using a silk-based hydrogel formulation for post tooth extraction wound management according to the embodiments of the present invention;

FIG. 2 is an optical absorbance graph of SF hydrogel with doxycycline at 550 nm;

FIG. 3 depicts scanning electron micrographs depicting the topological analysis of hydrogel samples (A) Silk hydrogel without doxycycline, (B) Doxycycline, and (C) SF hydrogel with doxycycline, Scale: 100 μm;

FIGS. 4A and 4B provide the FTIR spectroscopy patterns of lyophilized samples of silk fibers alone, doxycycline alone, and SF and doxycycline hydrogels;

FIG. 5 depicts photographs of zone of inhibition study of S. aureus with SF (A), doxycycline (B), and SF-doxycycline (C), and S. mutans with SF (D), doxycycline (E), and SF-doxycycline (F);

FIG. 6 provides a graphical representation of the cell viability by MTT assay in MC3T3, HDFC, MG63 cell lines in the presence of negative control, SF hydrogel, doxycycline alone, and SF-doxycycline hydrogel;

FIG. 7 provides graphical representation of drug release profile of silk alone, doxycycline alone, and SF-doxycycline hydrogel;

FIG. 8 provides a) image of rabbit's mouth indicating place of incisor extraction and SF-Doxycycline hydrogel was placement, b) image of rabbit's mouth indicating post operative suture, c) image of rabbit's mouth indicating healing of wound on the 7^{th} day; and

FIG. 9 depicts histopathological images of wounded tissue of rabbit groups on 14^{th} day after following treatments a) untreated or the control group, b) SF-Doxycycline hydrogel group, c) systemic antibiotic treated group, and d) vehicle control or the SF hydrogel group.

### DETAILED DESCRIPTION OF THE INVENTION

The present invention will now be described more fully hereinafter with reference to the accompanying drawings in which a preferred embodiment of the invention is shown. This invention may, however, be embodied in many different forms and should not be construed as being limited to the embodiment set forth herein. Rather, the embodiment is provided so that this disclosure will be thorough, and will fully convey the scope of the invention to those skilled in the art.

Silk is a high-quality natural fiber obtained from silk-spinning silkworms mainly composed of silk fibroin (SF) accounting for about 75%. Silk fibroin hydrogels are regarded as effective candidates for many applications due to their unique biocompatibility and biodegradability, especially in the field of biomedicine. Silk proteins are usually compounded with natural or synthetic polymers to prepare hydrogels. Further, silk fibroin hydrogels can be prepared by physical cross-linking and chemical cross-linking. Self-assembly of SF to hydrogel is known to be induced at 37° C. Further, the mixed solution of silk fibroin and sodium alginate (SA) allowed to stand at 37° C. leads to the self-assembly of the SF and SA molecules to form a hydrogel. Further, processes such as ultrasonication, shear action, electric filed effect, pH adjustment are known to initiate physical cross-linking to form hydrogels. Chemical treatments such as photopolymerization, High-intensity γ-rays irradiation, and use of agents such as genipin, glutaraldehyde, carbodiimide, and enzymes are known to initiate hydrogel formation. All these processes involve use of high temperature, expensive agents and procedures.

The present invention provides am extremely simple method to initiate hydrogel formation of SF embedded with antibiotic.

In one embodiment of present invention provides a silk-based hydrogel formulation for post tooth extraction wound management, as well as methods of making and using the same. The invention provides a novel silk-based hydrogel system for drug delivery tailored for sustained slow-release of drug molecules with antimicrobial properties and regenerative potential. In an embodiment the hydrogel is developed with silk fibroin protein by adding Doxycycline/Tetracycline and formed a “drug induced” hydrogel which will be lyophilised to remove the water molecule and increase the shelf life.

In another embodiment of the present invention, a novel silk-based hydrogel is provided capable of releasing antibacterial and osteoinductive agent at a sustained and controllable rate. Preferably, the prepared silk-based drug hydrogel releases high amount of drug for the first 3-5 days and showing low drug release for next 15 days.

In another embodiment of the present invention, the invention provides a silk fibroin-based hydrogel and antibiotic composition for wound healing of tooth extraction sockets, wherein, the silk fibroin-based hydrogel and antibiotic composition consists of 5% silk fibroin embedded with 0.4-20% antibiotic, more specifically, tetracycline or doxycycline, and the antibiotic used to prepare the silk fibroin-based hydrogel is super saturated solution of antibiotic, more specifically, 100% concentration (100 mg/ml) of antibiotic.

In another embodiment of the present invention, in order to prepare a novel silk-based hydrogel 5% silk fibroin solution and 500 ul of the solution in (16-21 degree Celsius) was taken. A super saturated solution of tetracycline/doxycycline (100 mg/ml) was made and vortexed for 30-60 sec (immediately after adding 100 mg of drug in 1 ml of DI water). The super saturated tetracycline/doxycycline solution was added in the 5% silk fibroin solution and hydrogel was formed immediately. The hydrogel was then stabilised in room temperature (16-21 degree Celsius) for 8 hours and kept in 4-6 degrees Celsius for 3 days (72 hours). Post refrigeration the gel is lyophilised and this lyophilised gel will be a dry hydrogel silk matrix with drug embedded in it. The lyophilised hydrogel will be showing release profile with high amount of drug release for 3-5 days and then low drug release for next 15 days.

Table 1. shows different ratios has been established of silk solution and drug solution tested for forming silk-based hydrogel, which are as follows:

TABLE 1

| | 5% Silk | 100 mg/ml | |
|---|---|---|---|
| S. No | Solution | drug solution | Hydrogelation |
| 1. | 500 ul | 80 ul | Yes |
| 2. | 500 ul | 60 ul | Yes |
| 3. | 500 ul | 40 ul | Yes |
| 4. | 500 ul | 20 ul | Yes |
| 5. | 250 ul | 40 ul | Yes |
| 6. | 250 ul | 30 ul | Yes |
| 7. | 250 ul | 20 ul | Yes |
| 8. | 250 ul | 10 ul | Yes |

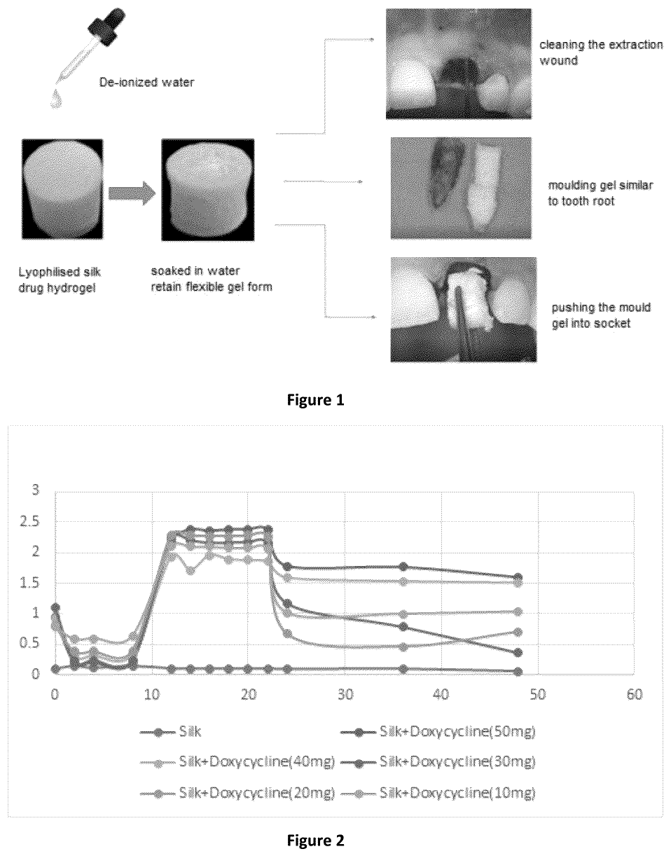

Referring to FIG. 1, is a schematic view of using a silk-based hydrogel formulation for post tooth extraction wound management according to the embodiments of the present invention. The lyophilised silk drug hydrogel can be placed in the extraction socket post tooth extraction where the hydrogel will be retained in the socket and embed in the blood clot. The hydrogel then locally releases the drug (tetracycline/doxycycline) in controlled manner in microbicidal concentration (localised concentration has to optimised: MIC is 1.2 ul/ml of body fluid) for 4-5 days and at sub-microbicidal concentration for next 15 days which will lead to host modulation activity (anti-collagenase activity of doxycycline/tetracycline) and enhance wound healing and osteo-induction.

### EXAMPLES

### Example 1

### Silk Purification

For extraction of silk fibroin, the cocoon was cut into small pieces and silkworm was removed. 2 grams of cocoon pieces were taken. Two 500 ml beaker was taken and 200 ml of DI water was taken, covered with aluminium foil and heated till boiling. 0.424 grams of sodium carbonate was taken and was added in both glass beaker with boiling water (to prepare a 0.02 M solution of Na2CO3) each, cut silk cocoon (2 g) was added into one of the beaker and kept under constant stirring (200 rpm) for 15 minutes. After 15 minutes silk fibroin was removed and was again added in other beaker with boiling DI water (0.02 M solution of Na2CO3) and kept for 15 minutes in same way as previous. After boiling for total of 30 minutes (15+15), silk fibroin was removed by spatula and was rinsed in cold water for 4-5 times to remove residual Na2CO3. Rest of the solution was discarded. Silk fibroin was squeezed well to remove water, spread in clean aluminium foil and kept in hot air oven at 60° C. overnight for drying. Stored it in clean aluminium foil. About 0.65-0.7 grams of silk fibroin was extracted by degumming 1 gram of silk fiber. 9.3 M LiBr solution was made to prepare a 20% (wt/vol) solution. (For every 1 gram of silk fibrion fibers 10 ml of 9.3 molar LiBr solution was made by adding 8.07 grams of LiBr and making up to 10 ml).

Silk fibroin (SF) fibers were kept in round bottom flask on hot water bath maintained at 60° C. Freshly prepared LiBr solution was made and poured immediately into the round bottom flask such that whole SF fiber submerged in it and kept under constant stirring at 200 rpm for 4 hrs. Once the SF completely dissolved it formed a transparent amber coloured solution. The transparent amber coloured solution of SF fibers were collected and put in dialysis membrane and tied so that it does not leak and was kept in 1 L flask filled with DI water for dialysis. Water was changed after 1 h, 4h, 12 h, 24 h, 36h, 48h, 60h, and 72h respectively. After dialysis was complete, the solution was taken out and extracted in a 50 ml falcon tube. This solution was then centrifuged at 9000 rpm for 20 minutes twice to remove visible impurities. The centrifuged sample (silk fibroin solution) was then collected and stored in 4° C.

Three sets of 100 ul of the solution was taken in pre-weighted small plastic boat and was kept for drying in hot air oven at 60° C. overnight. The dry weight of silk was calculated and was divided by 100 ul to get w/v % concentration of silk. Generally, 5% (w/v) of silk solution was obtained.

### Example 2

### Hydrogel Preparation

Silk Doxycycline Hydrogel was prepared by specific method which involved a precise combination of Silk fibroin protein, Supersaturated doxycycline solution at specific temperature and timed process.

500 ul of 5% silk fibroin solution was placed in 2 ml eppendroff tube (16-24° C.) and added 100-20 ul of freshly prepared supersaturated doxycycline hyclate (1 ml of 100 mg/ml doxycycline prepared within 1 min and thoroughly vertexed and added to prepared Silk Fibroin solution).

The regenerated Silk Fibroin-Doxycycline hydrogel started forming and was placed at room temperature for 8 Hour, followed by keeping at 4° C. for 3 days and then freezing at −80° C. Hydrogels were then lyophilized.

TABLE 2

| Drug Volume (μl) of | | | | |
|---|---|---|---|---|
| (100 mg/ml concentration | Drug | Drug | Silk 5% | Silk Dry |
| drug solution) | (mg) | (μg) | (μ) | (mg) |
| 100 | 10 | 10000 | 500 | 25 |
| 80 | 8 | 8000 | 500 | 25 |
| 60 | 6 | 6000 | 500 | 25 |
| 40 | 4 | 4000 | 500 | 25 |
| 20 | 2 | 2000 | 500 | 25 |

SF formed a gel like structure immediately after adding the doxycycline solution. Hydrogel formed is formed with no agitation. Initial matrix of hydrogel formulation happens immediately and after 8 hours the hydrogel matrix formulation is stabilized as been found in optical stability study.

### Example 3

### Gelation Kinetics

SF-doxycycline hydrogels were prepared with different final concentrations of doxycycline (10-, 20-, 30-, 40-, and 50-mg/ml) in a 96-well plate. OD measurements were taken at 550 nm in a multiplate reader (Tecan-Infinite M-200Pro).

Dynamics of the hydrogels was determined by measuring the absorbance at 550 nm. As shown in FIG. 2, after 8 hours there was an increase in the absorbance of all the SF-doxycycline hydrogels with the highest absorbance in the SF hydrogel with 50 mg doxycycline. The SF hydrogel with 40 mg doxycycline showed the least absorbance. SF hydrogel with no doxycycline showed no absorbance. The absorbance was stable (Platue in the graph) for 8-20 hours which indicate there was no matrix formation or change happened indicating SF-Doxy hydrogel stabilization, and The SF-Doxy hydrogel matrix formation is stabilized by 8-12 hours.

### Example 4

### a) Water Absorption and Swelling Study

Lyophilized hydrogels were weighed (Wₒ) and immersed in double-distilled water at 37° C. for different time intervals, starting from 0 h to 90 h. The swollen hydrogels were then weighed (Wₜ) and swelling ratio (SR) was calculated from the Equation 1.

### Equation 1. Swelling Ratio (SR) Calculation

$$SR = \frac{{W}_{t} - {W}_{o}}{{W}_{o}}$$

Where, Wₜ is the mass of the swollen get at time t and Wₒ is the mass of the dry gel at time 0⁹.

There was an increase in the net weight and swelling ratio of the hydrogel for upto 4 hours until the hydrogel reached the equilibrium. After 4 hours, net weight and swelling ratio started decreasing (washout of drug while continuous wetting). No change in the dimension of the hydrogel was seen for up to 12 hours as shown in Table 3. Dry weight was 25 mg.

TABLE 3
SF Doxycycline Hydrogel water absorption and swelling study

| | Dry | Wet | Swelling | Dimensions | Dimensions |
|---|---|---|---|---|---|
| Time | Weight | weight | ratio | dry (mm) | wet (mm) |
| (hours) | (mg) | (mg) | (%) | diameter | Diameter |
| 0 | 25 | 50 | 1 | 9 | 9 |
| 1 | 25 | 52 | 1.08 | 9 | 9 |
| 2 | 25 | 55 | 1.2 | 9 | 9 |
| 3 | 25 | 55 | 1.2 | 9 | 9 |
| 4 | 25 | 55 | 1.2 | 9 | 9 |
| 6 | 25 | 50 | 1 | 9 | 9 |
| 12 | 25 | 50 | 1 | 9 | 9 |

b) Optical Absorbance of SF Hydrogel with Doxycycline:

Dynamics of the hydrogels was determined by measuring the absorbance at 550 nm. As shown in FIG. 2, after 8h there was an increase in the absorbance of all the SF-doxycycline hydrogels with the highest absorbance in the SF hydrogel with 50 mg doxycycline. The SF hydrogel with 40 mg doxycycline showed the least absorbance. SF hydrogel with no doxycycline showed no absorbance. The SF-Doxy hydrogel matrix formation is stabilized by 8-12 hours.

### Example 5

### Topological Analysis by Scanning Electron Microscopy

Lyophilized samples were affixed to the scanning electron microscopy (SEM) sample holders using carbon tape. The hydrogels and doxycycline were coated with gold-palladium (Au—Pd; 80:20) using a sputter coater. The samples were examined in a HITACHI (S3700N) scanning electron microscope (SEM) at an accelerating voltage of 20 kV, using the SE detector. Micrographs were taken at magnification of ×500.

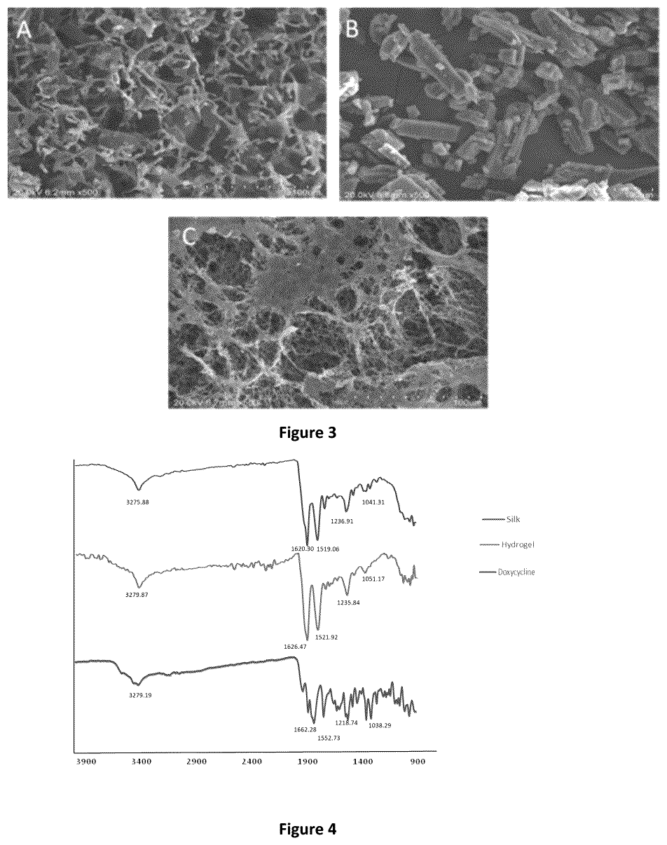

As shown in SEM image of FIG. 3A, the SF hydrogel had porous leaf-like structure. As shown in SEM image of FIG. 3B, doxycycline showed irregular cylindrical and cubical morphologies. As shown in SEM image of FIG. 3C, SF-doxycycline hydrogel showed fibrous and porous structure.

### Example 6

### Fourier-Transform Infrared Spectroscopy (FTIR) Spectroscopy

Lyophilized samples were analyzed by FTIR spectroscopy. The spectra were smoothened with constant smooth factor for comparison. FIG. 4 provide the FTIR spectroscopy patterns of lyophilized samples of silk fibers alone, doxycycline alone, and SF and doxycycline hydrogels.

### Silk:

FT-IR spectra of silk protein standard showed, the characteristic vibration bands around 3279.88 cm-1 shows N—H and O—H stretching 1620 cm-1 were assigned to the absorption peak of the peptide backbone of amide I (C═O stretching), bands around 1519 cm-1 to amide II (N—H bending), the bands around 1236.91 cm-1 to amide III (C—N stretching)

### Doxycycline:

FT-IR spectra of doxycycline standard showed, the characteristic vibration bands around 3279.19 cm-1 shows N—H and O—H stretching 1662.28 cm-1 were assigned to the absorption peak of the peptide backbone of amide I (C═O stretching), bands around 1552.73 cm-1 to amide II (N—H bending), the bands around 1216.74 cm-1 to amide III (C—N stretching)

### Silk and Doxycycline Hydrogel:

The combination of silk and doxycycline hydrogel shows the similar peaks compared to the control silk protein and doxycycline.

FT-IR spectra of silk and doxycycline hydrogel showed, the characteristic vibration bands around 3279.87 cml and shows N—H and O—H stretching at 1620.30 cm 1 which were assigned to the absorption peak of the peptide backbone of amide I (C═O stretching), bands around 1521.92 cm-1 to amide II (N—H bending), the bands around 1235.84 cm-1 to amide III (C—N stretching)

### Example 7

### X-Ray Diffraction

Diffraction pattern of the lyophilized samples were measured using an X-ray diffractometer XRD (Platinum ATR-Alpha II) operating at 40 kV, 30 mA and scan speed of 2° per minute. The diffraction index was recorded at 10-80°. Figure not provided.

### Example 8

### Biological Study

### a) Zone of Inhibition

S. aureus and S. mutans were cultured in nutrient broth and spread onto agar plates. Hydrogels (25 mg SF doxy hydrogel having 5 mm diameter with 2 mg drug) were kept in each plate in the center on the media and for doxycycline, drug was added in the center where the media was punched out. Plates were incubated at 37° C. for 24 h. After the incubation period, radius of the zone of inhibition was measured in each plate.

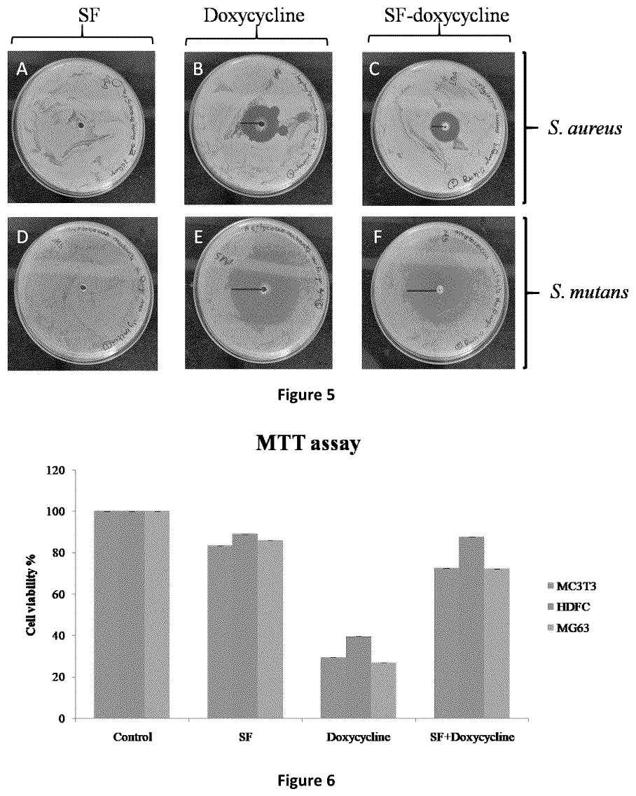

As shown in FIGS. 5A and 5D, S. aureus and S. mutans showed no zone of inhibition SF alone. Further, as depicted in FIGS. 5B and E, with doxycycline, zone of inhibition was 10 mm and 25 mm in S. aureus, and S. mutans, respectively. As depicted in FIGS. 5C and 5F, with SF-doxycycline, the zone of inhibition was less compared to only doxycycline. In S. aureus it was 7 mm, while in S. mutans it was 21 mm indicating active doxycycline in SF-doxycycline hydrogel and restricted release of doxycycline. This clearly shows the controlled release of the drug from the SF hydrogel.

### b) MTT (3-[4,5-Dimethylthiazol-2-Yl]-2,5 Diphenyl Tetrazolium Bromide) Assay

All the three cell lines (10,000 cells/200 μl medium) were cultured in their respective media in 96-well plates until they reached 60% confluency. Hydrogels and doxycycline were added in their respective wells. Plates were incubated at 37° C., 5% CO2, for 24 h. Media were removed and 200 μl of MTT solution (0.5 mg/ml prepared in phosphate buffer saline (PBS, Himedia, Catalog #TS1119)) was added to each well. Plate was incubated in dark at 37° C., 5% CO2 for 4h. MTT solution was then removed and 100 μl demthyl sulfoxide (DMSO) was added to each well to dissolve the formazan crystal. Absorbance was calculated at 570 nm in the multiplate reader. Percent of cell viability was calculated using Equation 2.

### Equation 2. Cell Viability Percentage Calculation

$$Cell viability \% = ( \frac{O . D . of treated cells}{O . D . of control cells} ) \times 100$$

Where, O.D. is optical density at 570 nm.

As shown in FIG. 6, all the three cell lines, MC3T3, HDFC, and MG63 (3 different cell lines were studied) showed least viability (40%) with doxycycline plain. Whereas, SF-doxycycline hydrogel showed cell viability around 75%-80% which is similar to cell viability with Silk Fibroin solution as compared to control. This clearly indicates that SF-doxycycline hydrogel is biocompatibility as compared to the drug alone.

In study in gingival fibroblast, cell viability was 87% with SF-doxycycline hydrogel.

### c) Release Profile

Samples (25 mg SF doxycycline hydrogel containing 2 mg drug) were taken in Eppendorf tubes and 1 ml of 0.9% saline (artificial saliva) was added in each tube. The tubes were kept in an incubator (REMI, Catalog #RHI80) at 37° C. 100 μl of the sample was taken from each tube at different time intervals to measure the O.D. value at 275 nm using UV-spectroscopy (Shimadzu UV-2600). O.D. values were measured for up to 168 h and plotted into the graph.

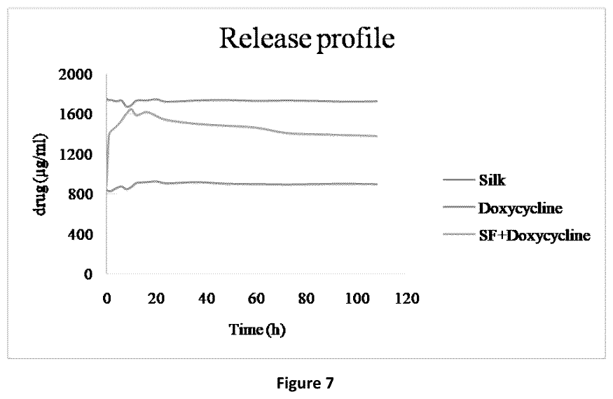

In SF-Doxycycline Hydrogel, the release profile was studied in Phosphate buffer solution. As depicted in FIG. 7, after initial burst of 20% of drug release in first hour, there was a sustained release of drug 70% for next 24 hours. Subsequently, the drug is released in a very slow and sustained manner for next 15 days. To imply with real scenario in dental wound, when hydrogel is inserted in the socket of tooth, there will be restricted release of doxycycline owing to semisolid nature of blood clot enclosing the hydrogel in the wound. Hence, the drug will be released in for more elongated time frame. The Initial drug release 20% will be released in first 3-4 hour, remaining 70% drug will be released in next 48-72 hours (3 days as per the routine systemic antibiotic/microbial concentration in localized wound) and then subsequent sub-microbial concentration release in next 15 days for host modulation and anti-collagenase activity (enhancing wound healing)

### d) Animal Study

Animal study was done in New Zeeland rabbit where incisor was extracted and SF-Doxycycline hydrogel was placed at the site of wound.

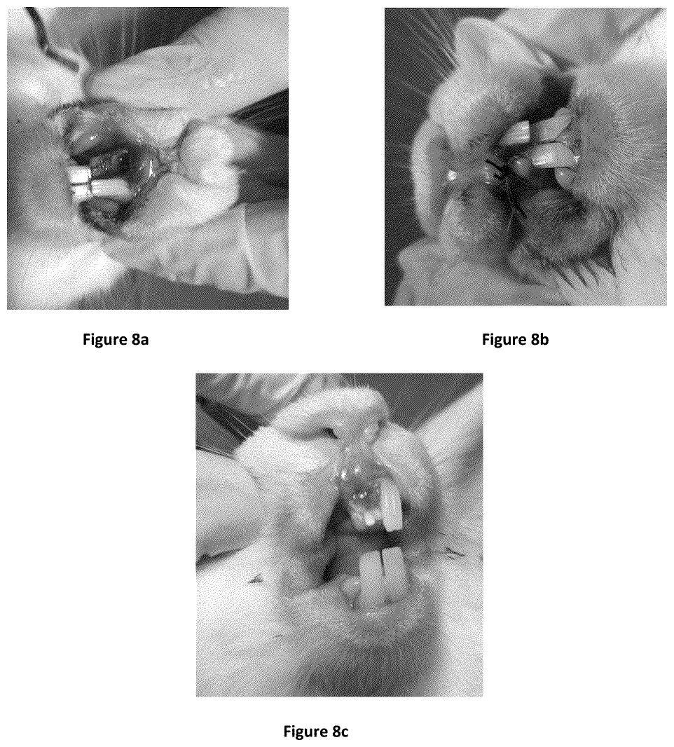

As depicted in FIG. 8a, the extraction of incisor in the mouth of rabbit created a wound and SF-Doxycycline hydrogel was placed at the site of the wound. FIG. 8b depicts post operative suture. After 7 days as depicted FIG. 8c, the wound was completely healed. This showed that SF-Doxycycline hydrogel worked efficiently in wound healing.

To further analyze four groups of rabbit were used where incisor was extracted creating a wound with the following treatments—a) Control—no antibiotic, b) SF—Doxycycline hydrogel group, c) antibiotic group—systemic antibiotic (4-5 mg doxycycline per day), and d) vehicle control—SF hydrogel. On 14^{th} day of treatment alveolar socket tissue, i.e. the tissue from the site of wound was taken from all the four groups and histopathological analysis was carried out.

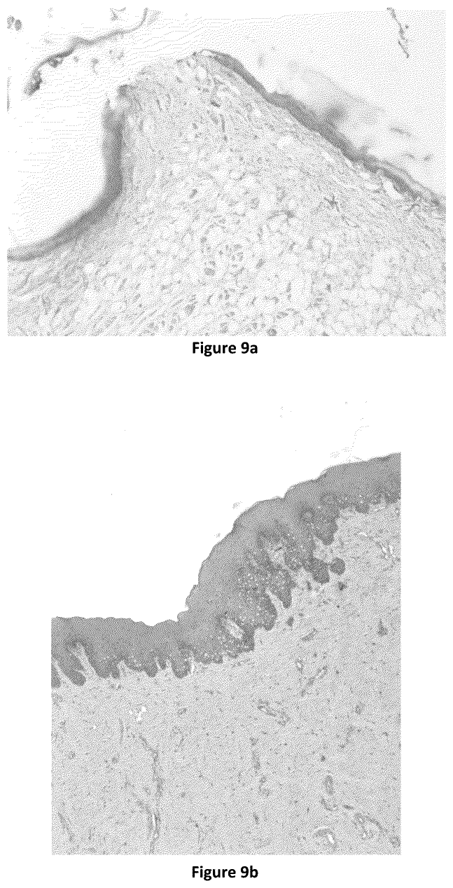

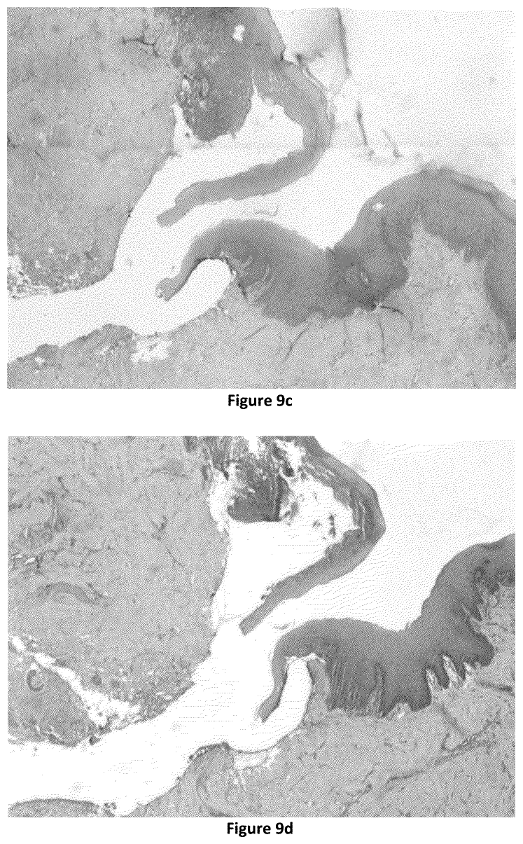

FIG. 9a depicts histopathological image of the untreated or the control group showing incomplete epithelial coverage on the superficial surface with chronic inflammatory cell infiltrate and budding endothelial cell proliferation in the lamina propria.

FIG. 9c depicts histopathological image of the systemic antibiotic treated group showing irregular epithelial stratification with variable thickness. The underlying lamina propria showed mild chronic inflammatory cell infiltrate.

FIG. 9d depicts histopathological image of the vehicle control or the SF hydrogel group showing irregular epidermal exterior surface. Lamina propria showed dense connective tissue stroma with mild inflammatory cell infiltrate and areas of Hemorrhage.

However, FIG. 9b depicts histopathological image of the SF-Doxycycline hydrogel group showing smooth uniform epidermal exterior surface. Lamina propria showed dense connective tissue stroma with mild inflammatory cell infiltrate. This clearly showed that the SF-Doxycycline hydrogel completely healed the wound compared to the all other 3 groups.

Also, gene expression studies indicated positive results for wound healing with SF-Doxycycline hydrogel group. bFGF, VEGF, TGF-gene expression was higher in SF-Doxycycline hydrogel group compared to systemic antibiotic group.

While certain exemplary embodiments have been described and shown in the accompanying drawings, it is to be understood that such embodiments are merely illustrative of, and not restrictive on, the broad invention, and that this invention not be limited to the specific constructions and arrangements shown and described, since various other changes. combinations, omissions, modifications and substitutions, in addition to those set forth in the above paragraphs, are possible. Those skilled in the art will appreciate that various adaptations and modifications of the just described embodiments can be configured without departing from the scope and spirit of the invention.

We claim:

1. A silk fibroin-based hydrogel and antibiotic composition for wound healing of tooth extraction sockets,
wherein,
the silk fibroin-based hydrogel and antibiotic composition consists of 5% silk fibroin embedded with 0.4-2% antibiotic, more specifically, tetracycline or doxycycline,
the antibiotic used to prepare the silk fibroin-based hydrogel is super saturated solution, i.e., 100% (100 mg/ml) concentration of antibiotic in de-ionized water, and
the silk fibroin-based hydrogel and antibiotic composition is formed by adding 100% (100 mg/ml) concentration of antibiotic to 5% silk fibroin for formation of hydrogel.

2. The silk fibroin-based hydrogel and antibiotic composition as claimed in claim 1, wherein, the composition enables high amount of drug release for the first 3-5 days, and subsequently shows a low drug release for next 15 days.

3. The silk fibroin-based hydrogel and antibiotic composition as claimed in claim 1, wherein, the composition enables local release of the antibiotic in a controlled manner in microbicidal concentration of MIC 1.2 ul/ml of body fluid for first 4-5 days.

4. The silk fibroin-based hydrogel and antibiotic composition as claimed in claim 1, wherein, the composition comprises the antibiotic tetracycline or doxycycline.

5. A method for preparing a formulation of silk fibroin-based hydrogel and antibiotic composition for wound healing of tooth extraction sockets, the method of formulation comprising steps of:
a) preparing 5% silk fibroin solution and maintaining at 16-21° C.;
b) preparing a super saturated solution of antibiotic in de-ionized water, and vortexing it for 2-3 min to prepare a 100% concentration antibiotic solution;
c) adding saturated antibiotic solution in the 5% silk fibroin solution to form a silk fibroin-antibiotic-based hydrogel;
d) stabilizing prepared hydrogel at a temperature of 16-21° C. for first 8 hours; and refrigerating at 4-6° C. for next 72 hours
e) lyophilising the stabilized prepared hydrogel to form a dry silk fibroin-based hydrogel matrix with antibiotic embedded in it,
wherein,
the silk fibroin-based hydrogel and antibiotic composition consists of 5% silk fibroin embedded with 0.4-2% antibiotic, more specifically, tetracycline or doxycycline, and
the antibiotic used to prepare the silk fibroin-based hydrogel is super saturated solution, i.e., 100% concentration of antibiotic in de-ionized water.

6. The method as claimed in claim 5, wherein, the method provides silk fibroin-based hydrogel and antibiotic composition which enables high amount of drug release for the first 3-5 days, and subsequently shows a low drug release for next 15 days.

7. The method as claimed in claim 5, wherein, the method provides silk fibroin-based hydrogel and antibiotic composition enables local release of the antibiotic in controlled manner in microbicidal concentration of MIC 1.2 ul/ml of body fluid for first 4-5 days.

8. The method as claimed in claim 5, wherein, the method provides silk fibroin-based hydrogel and antibiotic composition comprises the antibiotic tetracycline or doxycycline.
